(19) United States
(12) Patent Application Publication
(10) Pub. No.: US 2008/0097252 A1
(43) Pub. Date: Apr. 24, 2008

(54) Ultrasound and Pressure Therapy Wound Care Device

(76) Inventor: Eilaz Babaev, Minnetonka, MN (US)

Correspondence Address: Bacoustics, LLC, 5929 BAKER ROAD, SUITE 470, MINNETONKA, MN, 55345, US

(21) Appl. No.: 11/467,225

(22) Filed: Aug. 25, 2006

(51) Int. Cl. A61N 7/00

(52) U.S. Cl. 601/2

(57) ABSTRACT

The present invention is directed towards an apparatus and method for using a wound treatment device combining ultrasound therapy and pressure therapy. The device comprises an outer pressure chamber, ultrasonic transducers mounted on and/or within the sides of the pressure chamber, and treatment chamber located within the pressure chamber. The treatment chamber applies ultrasound induced topical pressure therapy to an injured extremity as to clean the wound, increase the flow positive healing elements to the wound, and/or deliver various therapeutic compounds to the wound. A treatment solution is held within, and may be circulated through, the treatment chamber. The pressure chamber surrounding the treatment chamber applies external pressure therapy to the injured extremity as to increase circulation within injured extremity. The ultrasound transducers mounted on and/or within the sides of the pressure chamber deliver therapeutic ultrasonic energy, through a coupling medium held within the pressure chamber, to the treatment chamber as to induce topical pressure therapy within the treatment chamber.

### BACKGROUND OF THE INVENTION

1. Field of the Invention

The present invention is directed towards an apparatus and method of wound treatment combining ultrasound therapy and pressure therapy.

2. Description of the Related Art:

Chronic wounds in the extremities encountered in clinical practice can be slow to heal and difficult to manage. Such wounds are often seen in diabetics, the elderly, and individuals with compromised or diminished peripheral circulation. Such wounds disable the patient, thereby reducing the patient's quality of life. An unhealed wound's susceptibility to infection increases a patient's morbidity and mortality. Placing the patient in an environment abundant in drug resistant infectious agents, such as hospital or institutional settings, further increases the patient's morbidity and mortality. Treating such wounds, especially after a serious infection has set in, burdens healthcare providers by increasing the time and resources that must be devoted to a single patient.

Maintaining a wound in a moist state, free of infections with a good blood supply and the correct balance of anti-inflammatory drugs is considered to be the ideal treatment to promote healing. (Jones et al. 2005) Attempting to create the ideal treatment, medical device manufactures and inventors have created a variety of devices utilizing topical negative pressure therapy or ultrasound.

Topical negative pressure therapy applies a controlled negative pressure to the surface of the wound. Generally, the negative pressure is created by a vacuum pump or similar mechanism. Represented devices are encompassed in U.S. Pat. No. 7,070,584 to Johnson et al.; U.S. Pat. No. 7,004,915 to Boynton et al.; U.S. Pat. No. 6,994,702 to Johnson; U.S. Patent No. 6,695,823 to Lina et al.; and U.S. Pat. No. 6,135,116 to Vogel et al. Topical negative pressure therapy devices have been shown to increase the flow of positive healing elements, such as, but not limited to, blood, nutrients, oxygen, and growth factors, to the wound and the rate of granulation, or tissue growth, while decreasing the level of bacteria and inflammatory agents present. Topical negative pressure therapy, however, has several limitations. Ineffective in treating sloughy or grossly infected wounds, topical negative pressure therapy devices are only capable of promoting healing in clean and debrided wound beds. (Jones et al. 2005) Furthermore, negative pressure therapy is contraindicated over necrotic tissue (Jones et al. 2005), the presence of which can hinder or prevent healing. High rental costs and expensive silver dressings further limit the applicability of topical negative pressure devices in wound care. This is especially true in light of the fact that 4 to 6 weeks of continuous therapy is required, during which time the machine cannot be used on more than one patient.

Re-injuring the wound when the dressings are changed further limits topical negative pressure therapy devices. The dressings employed by such devices are porous by necessity. As the wound heals, new tissue grows into the porous openings of the dressing. When the dressing is removed, healed tissue is removed with it.

Delivering ultrasonic energy through atomized liquid coupling mediums, ultrasonic wound care devices treat wounds by increasing the flow of positive healing elements to the wound. Represented devices are encompassed in U.S. Pat. No. 7,025,735 to Soring et al.; U.S. Pat. No. 6,964,647 to Babaev; U.S. Pat. No. 6,960,173 to Babaev; U.S. Pat. No. 6,916,296 to Soring; U.S. Pat. No. 6,761,729 to Babaev; U.S. Pat. No. 6,723,064 to Babaev; U.S. Pat. No. 6,663,554 to Babaev; U.S. Pat. No. 6,623,444 to Babaev; U.S. Pat. No. 6,601,581 to Babaev; U.S. Pat. No. 6,569,099 to Babaev; U.S. Pat. No. 6,533,803 to Babaev; and U.S. Pat. No. 6,478,754 to Babaev. Lacking relatively immediate contact with the target wound, these devices provide an inefficient transfer of ultrasound energy to the wound. Consequently, the ability of these devices to clean the wound, remove necrotic tissue, or destroy infectious agents is limited.

U.S. Pat. No. 5,305,737 to Vago and U.S. Pat. No. 5,665,141 to Vago discloses an ultrasound bath capable of treating an injured animal. Immersing the animal in a degassed coupling medium (working fluid) and transmitting ultrasound energy to the animal through the degassed coupling medium the bath treats the injured animal. Use of a degassed coupling medium prevents the formation of cavitations within the coupling medium.

In efforts to enhance the healing abilities of negative pressure therapy and ultrasound therapy, a device for treating wounds utilizing ultrasound induced topical pressure therapy has been developed by the author of the present application and disclosed in application Ser. No. 11/474,965 entitled “Ultrasound Wound Care Device and Method”. Ultrasonically inducing negative and positive pressure over the surface of a wound, ultrasound induced topical pressure therapy assists wound healing. Ultrasonic energy emitted into a treatment solution in contact with the wound induces cavitations within the treatment solution, leading to the formation of gas bubbles within the coupling medium. This phenomenon is similar to water boiling, but is not the result of heating the treatment solution. As gas bubbles form and dissipate against the surface of the wound micro domains of topical positive and negative pressure are created over the wound's surface. The alternating pressure removes necrotic tissue and other contaminates from the wound, while increasing the flow of positive healing elements to the wound.

Increasing blood flow to the site of the wound expedites healing by increasing the amount of positive healing elements delivered to the injury. Negative pressure therapy devices, even those employing the healing benefits of ultrasound, are only capable of increasing blood flow at the injury site. Circulation through the injured limb remains unaffected. Consequently, the amount of positive healing factors available to the wounded tissue is limited by the amount of positive healing factors entering the wound extremity. Various devices and methods exist for increasing blood flow within the extremities (i.e. arms, legs, feet, and hands). Applying alternating or fluctuating external pressure to an extremity increases circulation within the extremity (hereafter referred to as “external pressure therapy”). Devices employing such external pressure therapy are represented by U.S. Pat. No. 4,343,302 to Dillon, U.S. Pat. No. 4,269,175 to Dillon, and U.S. Pat. No. 3,961,625 to Dillon. Peripheral blood flow may also be enhanced by mechanical stimulation or agitation of the extremity as to work blood through the peripheral vascular system. Devices utilizing agitation to increase peripheral circulation are well known to the art. Though effective in increasing peripheral circulation, the mentioned devices are not capable of directly treating injuries or wounds located on the treated extremity.

Therefore, a need exists for a device and method capable of increasing the general circulation within a wounded extremity while simultaneously applying ultrasound induced topical pressure therapy to the wound.

### SUMMARY OF THE INVENTION

The present invention is directed towards a wound treatment device combining ultrasound therapy and pressure therapy. The device comprises an outer pressure chamber, ultrasonic transducers mounted on and/or within the sides of the pressure chamber, and a treatment chamber located within the pressure chamber. The treatment chamber applies ultrasound induced topical pressure therapy to an injured extremity as to clean the wound, increase the flow of positive healing elements to the wound, and/or deliver various therapeutic compounds to the wound. Cleaning a wound refers to debriding the wound, removing necrotic tissue from the wound, removing contaminants from the wound, and/or killing and removing bacteria, fungi, viruses, and/or other infectious microorganisms within the wound. A treatment solution is held within, and may be circulated through, the treatment chamber. The pressure chamber surrounding the treatment chamber applies external pressure therapy to the injured extremity as to increase circulation within injured extremity. The ultrasound transducers mounted on and/or within the sides of the pressure chamber deliver therapeutic ultrasonic energy, through a coupling medium held within the pressure chamber, to the treatment chamber as to induce topical pressure therapy within the treatment chamber.

The pressure chamber comprises a port or plurality of ports through which a coupling medium may be injected and/or evacuated. Injecting coupling medium into the pressure chamber increases the pressure within the pressure chamber. Inversely, evacuating coupling medium from the pressure chamber reduces the pressures within the pressure chamber. The coupling medium held within the pressure chamber may be any fluid capable of carrying ultrasound waves. Injecting and/or evacuating the coupling medium may be accomplished by a pump or plurality of pumps in communication with the ports. Alternating the pressure within the pressure chamber by repetitively injecting and evacuating the coupling medium may create a pumping action within the vasculature of the treated extremity. The induced pumping action may facilitate an increase in circulation within the treated extremity. The increased circulation allows for the delivery of more positive healing elements to the wounded extremity, consequently making more available to the wound(s) being treated.

In a possible alternative embodiment, the coupling medium may be injected into the pressure chamber through the ports of the pressure chamber as a pressurized stream. Injecting coupling medium as a pressurized stream allows direct pressure to be targeted to specific and/or alternating regions of the extremity being treated. Incorporating a nozzle tapered at its distal end into the ports may be done as to better target and/or create a pressurized stream of coupling medium. To facilitate the selective targeting of such pressurized streams the ports may comprise means of opening and/or closing the port, such as, but not limited to, valves or solenoids. Incorporating a means of selectively opening and closing the ports, such as, but not limited to a computer or control panel, into the treatment device of the present invention would allow the user to better direct pressurized streams of coupling medium as well as control the rate of pressure changes within the pressure chamber. The ports may also incorporate ultrasound transducers.

In another possible embodiment, the present invention may further comprise a manifold in communication with the ports possessing a series of valves, solenoids, and/or similar means of moving and directing the flow of coupling medium to and/or from various ports. The flow of fluid through the manifold and ports may be controlled by a computer, control panel, or similar means.

The alternating positive and negative pressure created within the pressure chamber by the sequential injection and evacuation of coupling medium creates an alternating negative and positive pressure within the treatment chamber. The treatment chamber comprises pliable sides capable of expanding and contracting in response to pressure changes within the pressure chamber, thereby coupling the pressure within the pressure chamber to that within the treatment chamber. The negative pressure created within the treatment chamber, as coupling medium is evacuated from the pressure chamber, creates an environment similar to conventional negative pressure therapy. A treatment solution may be circulated through the treatment chamber as to irrigate the wound while allowing for the evacuation of infectious microorganisms, inflammatory agents, and/or various contaminants removed from the wound out of the treatment chamber.

Ultrasonically inducing topical pressure therapy over the surface of a wound, the present invention treats wounds and assists wound healing. Ultrasonic energy emitted from the ultrasound transducers, located within and/or upon the walls of the pressure chamber, induces cavitations within the treatment solution held in the treatment chamber, leading to the formation of gas bubbles within the treatment solution. This phenomenon is similar to water boiling, but is not the result of heating the coupling medium. As gas bubbles form and dissipate against the surface of the wound micro domains of topical positive and negative pressure are created over the wound's surface. The alternating pressure may clean the wound.

The treatment solution within the treatment chamber may be liquid, gel, or similar fluid medium. Dissolving or suspending drugs within the treatment solution may be done to assist drug delivery during wound treatment. Liberating the dissolved or suspended drug from the coupling medium while inducing macro cavitations on the surface of the wound and micro cavitations along with micro streaming within the wound bed, the ultrasound waves transport the drug into and across the wound bed. The solution within the treatment chamber may also be capable of moistening the wound.

Within the wound, ultrasound waves induce micro cavitation and microstreaming. Killing bacteria and other infectious agents, the induced micro cavitation may disinfect the wound while cavitations within the coupling medium remove infectious agents from the wound. Inducing microstreaming within the wound bed, the delivered ultrasound waves may increase blood flow to the wound bed, thereby allowing for the increased delivery of positive healing elements to the wound and/or the removal of inflammatory agents from the wound. The fluctuating topical pressure may also help with the removal of inflammatory agents. Producing overlapping healing benefits, the fluctuating topical pressure and delivered ultrasound waves may exaggerate the actions of either when used alone, thereby creating a synergistic healing action.

The present invention may contain a plurality of ultrasound Langevin or cymbal transducers, preferably cymbal transducers. In such an arrangement, the selective activation of a single transducer and/or collection of transducers would allow ultrasound energy to be targeted to the area and/or areas of the treated extremity containing wound(s). Incorporating a means of selectively activating the ultrasound transducers within the treatment device, such as, but not limited to, a computer or control panel, allows the user to better target or direct ultrasonic energy emitted from the transducers to the wounds sought to treated.

The device may further comprise a means of mechanically agitating or vibrating the injured extremity as to further enhance of the therapeutic action of the external pressure chamber and/or internal treatment chamber. Agitating the system by shaking, vibrating, gyrating, oscillating, or inducing similar movement in the pressure chamber and/or treatment chamber may be done to induce a similar movement within the extremity being treated. The movement imparted in the extremity may enhance healing by further increasing circulation within the extremity being treated. Healing may be further enhanced by the induction of eddies or other turbulences within the treatment solution within the treatment chamber. Inducing eddies within the solution held in the treatment chamber may increase the flow and disbursement of infectious microorganisms, necrotic tissue, debris, contaminants, and/or inflammatory agents away from the wound being treated.

Treating a wound with the present invention does not require continued use of the device until the wound is healed. Rather, the present invention may be used intermittently to treat a patient's wound. After a patient has received a treatment session, the device can be cleaned and sterilized and then used to treat other patients. As to reduce the amount of cleaning between treatment sessions with different patients and to increase the versatility of the present invention, the treatment chamber may be removable and/or disposable and available in several configurations matching the extremity to be treated.

Though the present invention is primarily intended for the treatment of wounds, one skilled in the art should be able to immediately appreciate its application to the improvement of peripheral circulation in the absence of the wound care and to the treatment of injured, strained, and/or fatigued muscles, as wells as injured and/or inflamed joints.

One aspect of the present invention may be to treat wounds and assist wound healing.

Another aspect of the present invention may be to remove necrotic tissue, infectious agents, and other contaminants from the wound.

Another aspect of the present invention may be to deliver drugs to the wound.

Another aspect of the present invention may be to moisten the wound.

Another aspect of the present invention may be to disinfect the wound by killing bacteria and other infectious agents.

Another aspect of the present invention may be to increase the blood flow to the wound bed.

Another aspect of the present invention may be to increase the delivery of nutrients to the wound.

Another aspect of the present invention may be to remove inflammatory agents from the wound.

Another aspect of the present invention may be to create microdomains of fluctuating pressure over the surface of the wound being treated.

Another aspect of the present invention may be to provide topical pressure therapy.

Another aspect of the present invention may be to alternate pressure from positive to negative or negative to positive during treatment.

Another aspect of the present invention may be to create a synergistic relationship between ultrasound therapy and topical pressure therapy.

Another aspect of the present invention may be to flush out debris, necrotic tissue, bacterial, and other contaminants from the wound.

Another aspect of the present invention may be to allow for the simultaneous treatment of multiple patients with a single device.

Another aspect of the present invention may be to increase circulation to extremities.

Another aspect of the present invention may be to create a synergistic relationship between ultrasound therapy and external pressure therapy.

Another aspect of the present invention may be to create a synergistic relationship between ultrasound enhanced negative pressure therapy and external pressure therapy.

These and other aspects of the invention will become more apparent from the written descriptions and figures below.

### BRIEF DESCRIPTION OF THE DRAWINGS

FIG. 1 depicts a cross-section view of a wound treatment device combining ultrasound therapy and pressure therapy embodying the present invention.

FIG. 2 depicts a cross sectional view of a possible alternative embodiment in which a coupling medium is injected into the pressure chamber through the ports as a pressurized stream.

FIG. 3 depicts possible embodiment of a port which injects coupling medium as a pressurized stream.

### DETAILED DESCRIPTION OF THE INVENTION

The present invention is a wound treatment device combining ultrasound therapy and pressure therapy. Preferred embodiments of the present invention in the context of a device are illustrated in the figures and described in detail below.

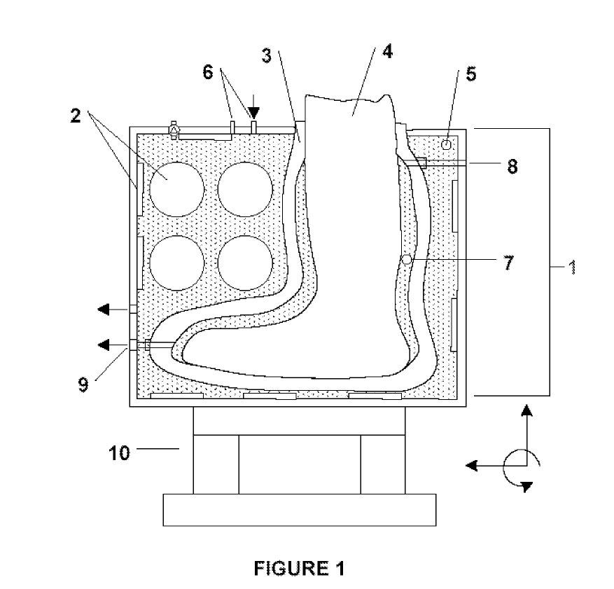

FIG. 1 depicts a cross-section view of a wound treatment device combining ultrasound therapy and pressure therapy embodying the present invention. The device comprises a pressure chamber 1, ultrasonic transducers 2 mounted on the sides (ceiling, floor, and/or walls) of the pressure chamber 2, and treatment chamber 3 located within pressure chamber 1. The extremity to be treated 4 is inserted into treatment chamber 3. The treatment chamber 3 applies ultrasound induced topical pressure therapy to an injured extremity facilitated by the delivery of ultrasound energy, emitted from the ultrasonic transducers 2, through coupling medium 5 to treatment chamber 3.

In keeping with FIG. 1, pressure chamber 1 comprises a plurality of ports 6 through which coupling medium 5 may be injected into and/or evacuated from pressure chamber 1. Injecting and/or evacuating the coupling medium may be accomplished by a pump or plurality of pumps (not shown) in communication with ports 6. Injecting coupling medium 5 into pressure chamber 1 increases the pressure within pressure chamber 1. Inversely, evacuating coupling medium 5 reduces the pressure within pressure chamber 1. Coupling medium 5 held within the pressure chamber may be any fluid capable of carrying ultrasound waves. As depicted in FIG. 1, coupling medium 5 may be injected and evacuated through different ports 6. Alternatively, coupling medium 5 may be injected and withdrawn through the same port(s). In such an embodiment, a means of for selectively injecting and/or extracting coupling medium 5 through port(s) 6 would be incorporated into the device. Such means for moving coupling medium may include, but are not limited to, a reversible pump.

In keeping with FIG. 1, treatment chamber 3 comprises a pliable shell capable of expanding and contracting in response to pressure changes within the pressure chamber, thereby coupling the pressure within the pressure chamber to that within the treatment chamber. The treatment chamber applies ultrasound induced topical pressure therapy to wounds on the extremity to be treated 4. Ultrasound transducers 2 deliver therapeutic ultrasonic energy, through coupling medium 5 held within the pressure chamber, to treatment chamber 3 as to induce topical pressure therapy within the treatment chamber. Alternating the pressure within treatment chamber 3 by repetitively injecting and evacuating coupling medium 5 from pressure chamber 1 creates a pumping action within the vasculature of the treated extremity. The induced pumping action facilitates an increase in circulation within the treated extremity. The increased circulation allows for the delivery of more positive healing elements to the wounded extremity, consequently making more available to the wound being treated. Treatment chamber 3 further comprises an opening at its apex through which the extremity to be treated 4 is inserted and a means of sealing the opening against the extremity to be treated 4. The sealing means may be, but is not limited to, an elastic or rubber band that presses the opening against the extremity to be treated, an adhesive that bonds the opening against the extremity to be treated, and/or a draw string. The sealing means prevents treatment solution 7 held within the treatment chamber 3 from escaping during the application of ultrasound induced topical pressure therapy and external pressure therapy. Treatment solution 7 within the treatment chamber may be liquid, gel, or similar fluid medium. Dissolving or suspending drugs within the treatment solution may be done to assist drug delivery during wound treatment. Possibly liberating the dissolved or suspended drug from treatment solution 7 while inducing macro cavitations on the surface of the wound and micro cavitations along with micro streaming within the wound bed, the ultrasound waves may transport the drug into and across the wound bed. Treatment solution 7 may be circulated through the treatment chamber 3. Circulating treatment solution 7 enters the treatment chamber through inlet 8 and exits through outlet 9. Circulating treatment solution 7 through the treatment chamber may be accomplished by a pump or plurality of pumps (not shown) in communication with the inlet 8 and/or outlet 9.

The treatment chamber depicted in FIG. 1 possesses a configuration similar to that of a sock. Alternatively, the treatment chamber may possess a configuration similar to a glove, sleeve, or boot. A sleeve confirmation allows the treatment of a leg or arm without treatment of the foot or hand respectively. In a sleeve embodiment of the present invention the treatment chamber would further comprise an opening at its base sealed against the extremity being treated. The treatment chamber may also possess alterative configuration, so long as the configuration chosen is capable of housing the extremity to be treated. The treatment chamber may be a permanently attached to the present invention or it may be removable and disposable.

A disposable treatment chamber allows the present invention to be used on sequential patients with minimal cleaning required between treatments.

In keeping with FIG. 1, the present invention may further comprise an agitating means 10 of mechanically agitating or vibrating the injured extremity as to further enhance of the therapeutic action of the external pressure chamber and/or internal treatment chamber. Agitating means 10 may be coupled to an external surface of the pressure chamber 1. Alternatively, agitating means 10 may be located within the pressure chamber. Agitating means 10 may include a shaker platform, magnetic stirrer or mechanical mixer and may agitate pressure chamber 1 and/or treatment chamber 3.

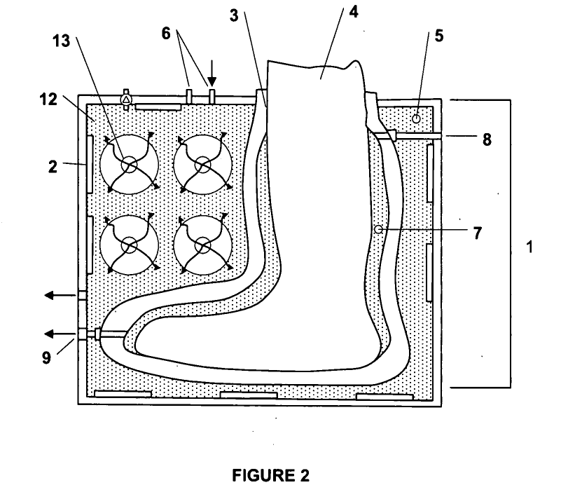

FIG. 2 depicts a cross sectional view of a possible alternative embodiment in which coupling medium 5 is injected into the pressure chamber 1 through port(s) 6 as a pressurized stream 12 through a nozzle 13. Injecting coupling medium as a pressurized stream allows direct pressure to be target to specific and/or alternating regions of the extremity being treated.

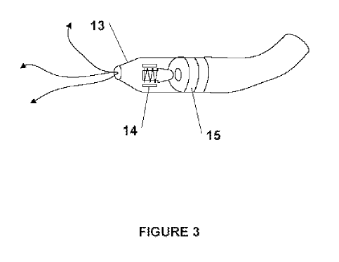

FIG. 3 depicts a possible embodiment of a port which injects coupling medium as a pressurized stream comprising a nozzle 13, which may be tapered at its distal end, a solenoid 14 allowing for the selective opening and closing of the port, and ultrasound transducer opening 15.

Nozzle 13 may allow for better targeting and/or creation of a pressurized stream of coupling medium. Solenoid 14 facilitates the selective targeting of such pressurized streams against a particular location of the extremity to be treated when multiple ports are employed. Selectively opening solenoid 14 of the port(s) emitting a pressurized stream at the desired region allows the user of the device to target the streams. Incorporating a means of selectively opening and closing the ports (not shown), such as, but not limited to a computer, valve and/or pump control panel, into the treatment device of the present invention would allow the user to better direct pressurized streams of coupling medium and/or control the rate of pressure changes within the pressure chamber. Incorporation of a port control means may also enable the user to coordinate the injection of pressurized streams from the port(s) enabling the creation of various spray patterns against the extremity to be treated.

The ultrasound waves emitted from transducers employed in the present invention may vary with respect to frequency; approximately 15 kHz to 20 MHz. The preferred low-frequency range is approximately 20 kHz-100 kHz. The more preferred low-frequency range is approximately 25 kHz-50 kHz. The recommend low-frequency is approximately 30 kHz. The preferred high-frequency ultrasound range is approximately 0.7 MHz-20 MHz. The more preferred high-frequency range is approximately 0.7 MHz-1 MHz. The recommend high-frequency is approximately 0.7 MHz. The ultrasound waves employed may also vary with respect to amplitude; approximately 1 micron and above. The preferred low-frequency amplitude is approximately 30 microns-100 microns. The recommended low-frequency amplitude is approximately 100 microns. The high-frequency amplitude can be 1 micron and above. The preferred high-frequency amplitude is approximately 5 microns. The recommended high-frequency amplitude is approximately 10 microns. Employing low frequency ultrasound waves is the preferred method of treatment.

Although specific embodiments have been illustrated and described herein, it will be appreciated by those of ordinary skill in the art that any arrangement that is calculated to achieve the same purpose may be substituted for the specific embodiments shown. It is to be understood that the above description is intended to be illustrative and not restrictive. Combinations of the above embodiments and other embodiments will be apparent to those having skill in the art upon review of the present disclosure. The scope of the present invention should be determined with reference to the appended claims, along with the full scope of the equivalents to which such claims are entitled.

I Claim:

1. An apparatus for treating extremities comprising:
a pressure chamber containing a coupling medium;
a treatment chamber within said pressure chamber;
said treatment chamber containing a treatment solution; and
at least one ultrasonic transducer for emitting ultrasound waves into said pressure chamber.

2. The device of claim 1, wherein the ultrasound waves emitted from said ultrasonic transducer comprise a frequency in the approximate range of 15 kHz-20 MHz.

3. The device of claim 1, wherein the ultrasound waves emitted from said ultrasonic transducer comprise a preferred low-frequency in the approximate range of 20 kHz-100 kHz.

4. (canceled)

5. The device of claim 1, wherein the ultrasound waves emitted from said ultrasonic transducer comprise a recommended low-frequency of approximately 30 kHz.

6. The device of claim 1, wherein the ultrasound waves emitted from said ultrasonic transducer comprise a preferred high-frequency in the approximate range of 0.7 MHz-20 MHz.

7. (canceled)

8. The device of claim 1, wherein the ultrasound waves emitted from said ultrasonic transducer comprise a recommended high-frequency of approximately 0.7 MHz.

9. The device of claim 1, wherein the ultrasound waves emitted from said ultrasonic transducer comprise an amplitude of at least 1 micron.

10. The device of claim 1, wherein the ultrasound waves emitted from said ultrasonic transducer comprise a preferred low-frequency amplitude in the range of approximately 30-250 microns.

11. The device of claim 1, wherein the ultrasound waves emitted from said ultrasonic transducer comprise a recommended low-frequency amplitude of approximately 50 microns.

12. The device of claim 1, wherein the ultrasound waves emitted from said ultrasonic transducer comprise high-frequency amplitude of at least 1 micron.

13. The device of claim 1, wherein the ultrasound waves emitted from said ultrasonic transducer comprise a preferred high-frequency amplitude of at least 5 microns.

14. The device of claim 1, wherein the ultrasound waves emitted from said ultrasonic transducer comprise a recommended high-frequency amplitude of approximately 10 microns.

15. The apparatus of claim 1 further, comprising a means of agitating said treatment chamber.

16. The apparatus of claim 1 further, comprising a means of selectively activating said ultrasonic transducer.

17. (canceled)

18. (canceled)

19. A pressure chamber for treating extremities comprising:
at least one port for injecting a coupling medium into and/or extracting a coupling medium from the pressure chamber; and
at least one pump in communication with said port;
wherein said pump moves said coupling medium into the pressure chamber through said port.

20. The pressure chamber of claim 19, further comprising a means for selectively moving coupling medium through said port.

21. The pressure chamber of claim 19, further comprising a means for selectively opening and/or closing said port.

22. A treatment chamber for treating extremities comprising:
a pliable shell with an apex;
an opening at said apex through which the extremity to be treated may be inserted; and
a means of sealing said opening about an extremity to be treated.

23. The treatment chamber of claim 22 further, comprising at least one inlet through which a treatment solution is moved through the treatment chamber.

24. The treatment chamber of claim 23 further, comprising at least one pump in communication with said inlet, wherein said pump moves the treatment solution through the treatment chamber.

25. The treatment chamber of claim 22, wherein at least a portion of the treatment chamber possesses a general configuration resembling a glove.

26. The treatment chamber of claim 22, wherein at least a portion of the treatment chamber possesses a general configuration resembling a sock.

27. (canceled)

28. (canceled)

29. The port of claim 19 for injecting and/or expelling a coupling medium comprising:
a nozzle attached to the port,
said nozzle targeting the coupling medium through the port to the treatment chamber,
at least one cymbal ultrasonic transducer within the nozzle,
a means for opening and/or closing the port.

30. (canceled)

31. (canceled)

32. The treatment chamber of claim 22 further comprising a pliable shell that is easily removable and disposable.
